(19) United States
(12) Patent Application Publication
(10) Pub. No.: US 2009/0163832 A1
(43) Pub. Date: Jun. 25, 2009

(54) DATA MANAGING DEVICE FOR A DIAGNOSTIC INSTRUMENT

(75) Inventor: Iqbal Sunderani, Unionville (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: BTNX INC., Markham (CA)

(21) Appl. No.: 12/338,857

(22) Filed: Dec. 18, 2008

Related U.S. Application Data
(60) Provisional application No. 61/015,339, filed on Dec. 20, 2007.

(51) Int. Cl. A61B 5/20; G05B 15/02; G05B 11/01; G06F 17/00

(52) U.S. Cl. 600/573; 700/83; 700/17; 700/90

(57) ABSTRACT

A data managing device for connection to a diagnostic instrument, having an outer casing which is sealed to prevent the entry of foreign substances. The data managing device includes a controller for controlling the operation of the data managing device, a casing for housing of the controller, a display in communication with the controller and sealed with respect to the casing, a user input coupled to the controller and sealed with respect to the casing, and a communications interface in communication with the controller for communication with the diagnostic instrument. The controller is configured to control the diagnostic instrument by communicating specific protocols to the diagnostic instrument. The display and user input may be a touchscreen.

### FIELD

Example embodiments described herein relate to data managing of diagnostic instruments and, in particular, to devices and methods for facilitating such data managing.

### BACKGROUND

A typical laboratory may have numerous diagnostic instruments for analyzing of sample specimen. In some of these diagnostic instruments, a test strip is treated with sample specimen and subsequently processed by inputting of the test strip into the instrument. Many diagnostic instruments are limited in their functionality, and may only be able to generate raw or limited data based on testing of the sample specimen. Examples of diagnostic instruments for respective sample specimen are urine analyzers, glucose monitoring devices, pregnancy testers, narcotic testers, and the like.

In some conventional applications, personal computer (PC) compatible software may be used for data management of diagnostic instruments. In such applications, a desktop PC, laptop, tablet, or the like would be required in order to use the software for control of the diagnostic instruments. Such applications may require installation of specific programs into a computer, which may be cumbersome to install, configure, and subsequently execute for each use, especially to technicians and the like in a laboratory setting. Another difficulty with these computer applications is that a typical testing facility may be surrounded by various laboratory specimens and potentially bio-hazardous chemicals. As such, care and cleaning of the keyboard, mouse and other parts and components of the device would be difficult to perform and maintain. In addition, the size of a typical desktop computer may be cumbersome in a limited spaced environment.

In other applications, some diagnostic instruments incorporate some data managing functionalities as a built-in feature of the diagnostic instrument itself. However, such systems may require existing users to replace or upgrade their diagnostic instruments to these versions, adding extra cost and potentially wasting any existing diagnostic instruments.

In addition, some patients may wish to perform their own diagnostic tests at home. This is especially the case where the patient requires diagnostic tests on a regular basis, for example urine analysis or glucose monitoring by patients with diabetes.

Accordingly, it may be advantageous to provide a device for facilitating data management in diagnostic instruments to address at least some of the deficiencies of the prior art.

### BRIEF DESCRIPTION OF THE DRAWINGS

Example embodiments will now be described by way of example with reference to the accompanying drawings, through which like reference numerals are used to indicate similar features.

FIG. 1 shows a block diagram of an example of a communications system to which example embodiments can be applied;

FIG. 2A shows, in diagrammatic form, a front view of an example data managing device to be used on the communications system shown in FIG. 1;

FIG. 2B shows a left side view of the data managing device of FIG. 2A;

FIG. 2C shows a right side view of the data managing device of FIG. 2A;

FIG. 3 shows a block operational diagram of the data managing device shown in FIG. 2A;

FIG. 4 shows an example conversation between the data managing device shown and a diagnostic instrument shown in FIG. 1;

FIG. 5A shows a diagrammatic view of an example graphical user interface for the data managing device of FIG. 2A, displaying a main menu for operation of a diagnostic instrument;

FIG. 5B shows a diagrammatic view of example icons which may be displayed in the user interface shown in FIG. 5A.

FIG. 6 shows a diagrammatic view of an example graphical user interface for the data managing device of FIG. 2A, displaying a testing menu for testing of sample specimen;

FIG. 7 shows a diagrammatic view of an example graphical user interface for the data managing device of FIG. 2A, displaying a worklist menu for facilitating testing of sample specimen;

FIG. 8 shows a diagrammatic view of an example graphical user interface for the data managing device of FIG. 2A, displaying testing instructions;

FIG. 9 shows a diagrammatic view of an example graphical user interface for the data managing device of FIG. 2A, displaying the results of a test;

FIG. 10 shows a diagrammatic view of an example graphical user interface for the data managing device of FIG. 2A, displaying a menu for a calibration test of a diagnostic instrument;

FIG. 11 shows the graphical user interface of FIG. 10, displaying results of a calibration test;

FIG. 12 shows a diagrammatic view of an example graphical user interface for the data managing device of FIG. 2A, displaying a menu for a quality control test of a diagnostic instrument;

FIG. 13 shows a diagrammatic view of an example graphical user interface for the data managing device of FIG. 2A, displaying results of a quality control test;

FIG. 14 shows a diagrammatic view of an example graphical user interface for the data managing device of FIG. 2A, displaying a menu for a proficiency test of a diagnostic instrument;

FIG. 15 shows a diagrammatic view of an example graphical user interface for the data managing device of FIG. 2A, displaying a menu for searching of test results;

FIG. 16 shows a diagrammatic view of an example graphical user interface for the data managing device of FIG. 2A, displaying a device menu;

FIG. 17 shows a diagrammatic view of an example graphical user interface for the data managing device of FIG. 2A, displaying a main menu for an administrator;

FIG. 18 shows a diagrammatic view of an example graphical user interface for the data managing device of FIG. 2A, displaying an operator configuration menu for an administrator;

FIG. 19 shows a diagrammatic view of an example graphical user interface for the data managing device of FIG. 2A, displaying a calibration configuration menu for an administrator;

FIG. 20 shows a diagrammatic view of an example graphical user interface for the data managing device of FIG. 2A, displaying quality control configuration menu for an administrator;

FIG. 21 shows a diagrammatic view of an example graphical user interface for the data managing device of FIG. 2A, displaying a menu for managing of quality control lots;

FIG. 22 shows a diagrammatic view of an example graphical user interface for the data managing device of FIG. 2A, displaying a menu for managing of control values in quality control lots; and

FIG. 23 shows a diagrammatic view of an example graphical user interface for the data managing device of FIG. 2A, displaying a menu for configuration of test strips,

### SUMMARY

The present application provides a data managing device for connection to a diagnostic instrument, having an outer casing which is sealed to prevent the entry of foreign substances.

According to one example embodiment is a data managing device for connection to a diagnostic instrument, the diagnostic instrument having an input component for receiving of sample specimen, an analyzing component for generating data based on testing of sample specimen, a communications component, the diagnostic instrument being responsive to specific protocols communicated through the communications component for controlling of the diagnostic instrument. The data managing device includes a controller for controlling the operation of the data managing device, a casing for housing of the controller, a display in communication with the controller and sealed with respect to the casing, a user input coupled to the controller and sealed with respect to the casing, and a communications interface in communication with the controller for communication with the communications component of the diagnostic instrument. The controller is configured to control the diagnostic instrument by communicating the specific protocols to the diagnostic instrument over the communications interface.

According to another example embodiment is a method of facilitating data management in a diagnostic instrument, the diagnostic instrument having an input component for receiving of sample specimen, an analyzing component for generating data based on testing of sample specimen, a communications component, the diagnostic instrument being responsive to specific protocols communicated through the communications component for controlling of the diagnostic instrument, the method comprising providing a data managing device for connection to the diagnostic instrument; storing at the data managing device the specific protocols of the diagnostic instrument; coupling the data managing device to the diagnostic instrument; and accessing, based upon the coupling of the data managing device to the diagnostic instrument, the specific protocols for controlling of the diagnostic instrument over the communications interface.

According to another example embodiment is a method of facilitating data management in a diagnostic instrument, the diagnostic instrument having an input component for receiving of sample specimen, an analyzing component for generating data based on testing of sample specimen, a communications component, the diagnostic instrument being responsive to specific protocols communicated through the communications component for controlling of the diagnostic instrument, the method comprising providing a data managing device for connection to the diagnostic instrument, the data managing device configured for providing instructions to the diagnostic instrument for testing of sample specimen; determining in the data managing device that control testing in the diagnostic instrument is required based on predetermined criteria; and disabling testing of sample specimen in the diagnostic instrument until control testing is completed.

### DETAILED DESCRIPTION

The terms “include” and “comprise” are used interchangeably within this document and are non-exhaustive when used, meaning, for example, that elements and items that are identified as including or comprising certain components or features can also include additional components or features that are not expressly identified.

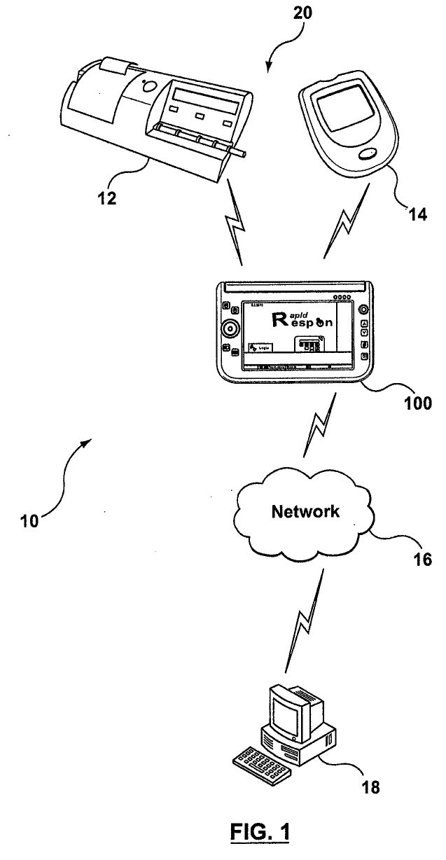

Reference is now made to FIG. 1, which shows a block diagram of a communications system 10 that in example embodiments facilitates the data managing and operation of diagnostic instruments 20. The communications system 10 includes at least one data managing device 100 which is in communication with the diagnostic instruments 20, for controlling of the diagnostic instruments 20. The data managing device 100 is also configured for communications with a network 16. A server device 18 may also be in communication with the data managing device 100, either directly or through the network 16. Generally, the data managing device 100 facilitates data managing and controlling functions of the diagnostic instruments 20. The data managing device 100 may be preconfigured to operate with the specific diagnostic instrument upon connection with the particular diagnostic instrument 20. The data generated from the diagnostic instruments 20 may subsequently be sent to the server device 18, for example for subsequent storage, further processing, or sending data results to a third party such as a regulatory agency (not shown).

The diagnostic instrument 20 may for example include an input component for receiving of sample specimen, an analyzing component for generating data based on testing of sample specimen, and a communications component. Generally, the diagnostic instrument 20 may be responsive to specific protocols communicated through the communications component for controlling of the diagnostic instrument 20. Examples of diagnostic instruments 20 include a urine analyzer 12, a glucose monitoring device 14, or other diagnostic instruments for analyzing of sample specimen. An example of a urine analyzer 12 that may be used in the system 10 is the “Rapid Response (TM) 120” available from BTNX Inc. An example of a glucose monitoring device 14 is the “Rapid Response (TM) Glucose Meter”, also available from BTNX Inc.

The network 16 may include any combination of a cellular or packet-based wireless network, a local area network, a wireless local area network, the Internet, an intranet or a direct connection between two devices. The network 16 may also include an enterprise network comprising an intranet for a facility such as a laboratory, hospital, corporation or other type of organization.

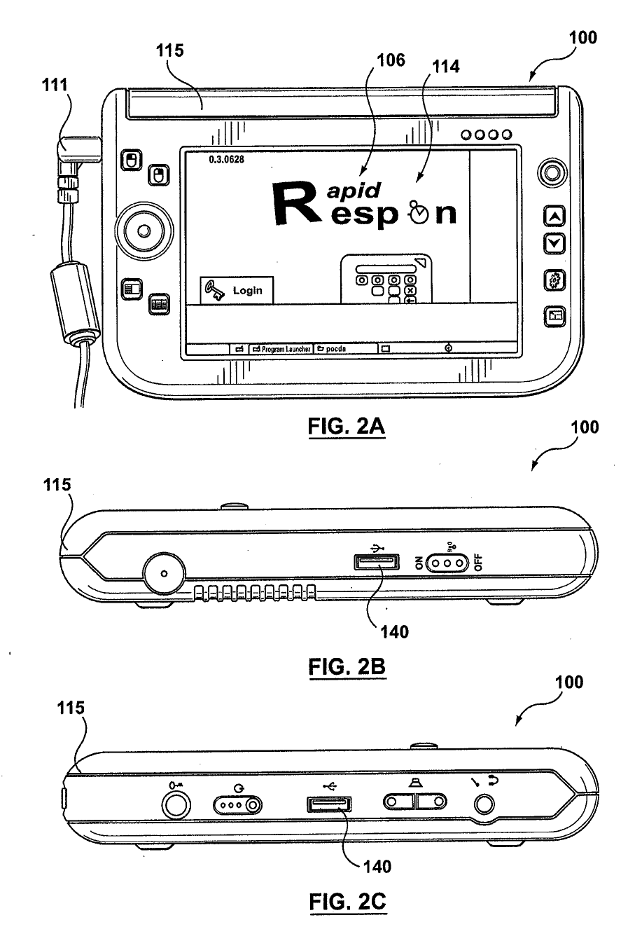

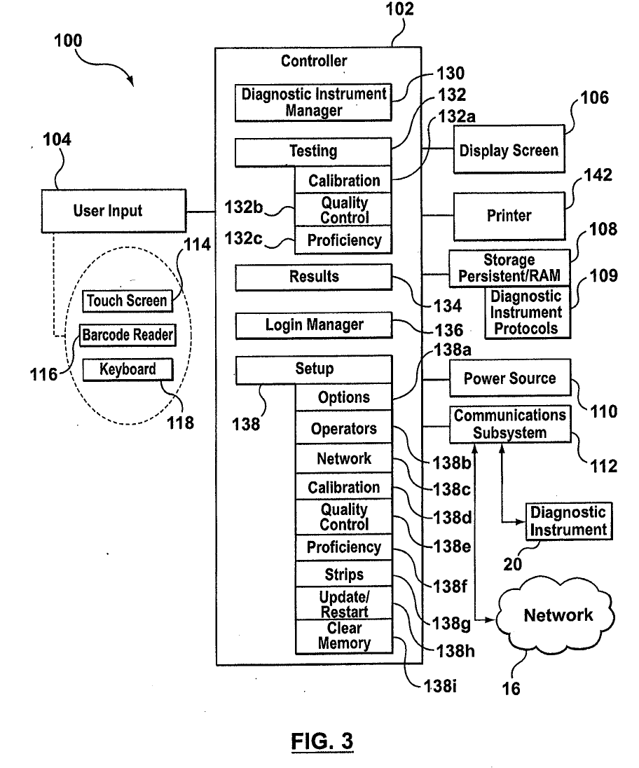

Reference is now made to FIGS. 2A-2C, wherein FIG. 2A shows a perspective view of the data managing device 100 to be used on the communications system 10 (FIG. 1), FIG. 2B shows a left side view of the data managing device 100, and FIG. 2C shows a right side view of the data managing device 100. As shown in FIG. 2A, the device includes a casing 115, and a display screen 106, which are sealed to prevent the entry of foreign substances such as laboratory specimens and potentially bio-hazardous chemicals. The casing 115 may be formed of any rigid material, and acts as a housing for the contents of the device 100. The display screen 106 may also include a touchscreen 114. As shown, the display screen 106 has a relatively flat surface, so that the surface of the device 100 may be wiped or otherwise cleaned, for example using a cloth. In other example embodiments, the casing 115 of the device 100 is hermetically sealed to further prevent air from entering therein. In some example embodiments, the device 100 is a portable handheld electronic device.

As shown in FIGS. 2B and 2C, the device 100 also includes a number of ports 140 for connection with the network 16 (FIG. 1), the diagnostic instruments 20, and other peripherals such as printers, removable keyboards or keypads, and the like. Example ports include Universal Serial Bus (USB), Communication ports (COM ports), serial ports, Ethernet, network ports, printer ports. As shown, the device 100 may also include a power outlet 111.

Reference is now made to FIG. 3, which shows in greater detail a block operational diagram of the device 100. The device 100 is in at least some example embodiments configured to be readily used with diagnostic instruments 20 by those who may wish to forego the usual installation and execution of computer software and/or are otherwise not computer savvy. Examples of such persons include laboratory technicians, nursing professionals, students, and patients administering their own diagnostic tests.

As shown in FIG. 3, the device 100 has a controller 102 for controlling operation of the device 100, a display screen 106, at least one user input 104, and a communications subsystem 112 coupled to the controller 100 for sending and receiving communications information over a network 16 or to the diagnostic instruments 20. The device 100 also includes electronic storage 108, which can include transient memory such as RAM and one or more persistent storage elements such as, but not limited to, flash memory or a hard drive. The controller 102 can include one or more microprocessors that are coupled to the persistent and/or transient memory of storage 108. Storage 108 stores information and software enabling the microprocessor(s) of controller 102 to implement the device 100 functionality described in greater detail below. The storage 108 may also include diagnostic instrument protocols 109 for controlling of the diagnostic instrument 20. The device 100 also includes a power source 110, for example power outlet 111 (FIG. 2A) or rechargeable battery (not shown) for providing power to the device 100. The device 100 may for example recharge the battery (not shown) at a charging station (not shown), through one of the ports 140 (FIG. 2A), or through power outlet 111 (FIG. 2A). A printer 142 may also be coupled to the controller 102 for printing of test results and the like. The printer 142 may be a conventional printer accessed through communication via one of the communication ports 140 (FIG. 2A) or may be integrated within the hardware of the device 100.

As shown, suitable options for the user input 104 may be a touchscreen 114, a barcode reader 116, and a keyboard 118. The barcode reader 116 may for example be integrated into the device 100 or connected through one of the ports 140 (FIG. 2A).

Referring again to FIG. 3, there are a number of modules of the controller 102 that may perform desired functions on the device 100. In one example embodiment, the modules on controller 102 are implemented by software applications running on a processor of the controller 102, the executable code for such applications being stored on storage 108. As shown, the controller 102 includes a diagnostic instrument manager module 130, a testing module 132, a results module 134, a login manager module 136 and a setup module 142. The testing module 132 includes a calibration module 132a, quality control module 132b and a proficiency module 132c. The setup module 138 includes an options module 138a, an operator configuration module 138b, a network module 138c, a calibration module 138d, a quality control configuration module 138e, a proficiency configuration module 138f, a strips configuration module 138g, an update/restart module 138h and a clear memory module 138i. In various embodiments, additional or fewer modules may be implemented by controller 102, and some or all of the functions performed by some modules could be combined into other modules or split into separate modules.

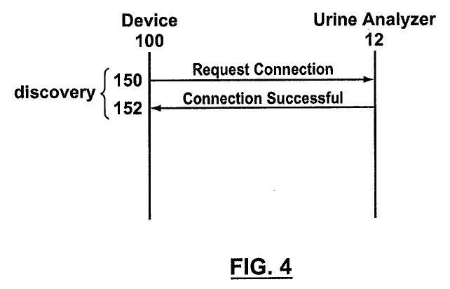

FIG. 4 shows an example conversation 200 between the device 100 and one of the diagnostic instruments 20 (FIG. 1), for example a urine analyzer 12. In some example embodiments, the conversation 200 is triggered upon connection between the device 100 and the diagnostic instrument 20. The conversation 200 may also be triggered by other means, such as restarting or resetting the device 100, logging out of the device 100, etc. Generally, the conversation 200 permits the device 100 to determine the presence of the particular diagnostic instrument 20, for accessing of the appropriate protocols specific to the diagnostic instrument 20. Although the example conversation 200 shown is with a urine analyzer 12, it can be appreciated that the conversation 200 may be modified as appropriate depending on the particular diagnostic instrument 20. As shown, at step 150, the device 100 sends a Request Connection command 150 to the urine analyzer 12. In response the urine analyzer 12 sends a Connection Successful command 152 back to the device 100. After receiving the Connection Successful command 152, the device 100 can access the appropriate protocols from the diagnostic instrument protocols 109 (FIG. 3), for controlling of the urine analyzer 12. The protocols may for example be any suitable communication protocol such as serial-based communications, for example based on the System.IO.Ports.Port class in the .NET Library. Accordingly, in some example embodiments, a user may operate the urine analyzer 12 by connecting the device 100 without further installation and subsequent execution of software, etc.

After connection of the device 100, the display screen 106 displays a login interface (not shown) for logging into the device 100. The login functions may for example be controlled by the login manager module 136 (FIG. 3). An operator or an administrator may log in. An operator includes laboratory technicians, nursing professionals, students, patients administering their own diagnostic tests, and the like. An administrator includes medical professionals, lab supervisors, teachers, systems administrators, and the like. A user, which includes either an operator or an administrator, may for example login by inputting login information using a keyboard 118 or touchscreen 114, by scanning an identification badge containing a barcode (not shown) with the barcode reader 116, or by any other suitable means (such as fingerprint and other biometric scanners, etc.). For convenience, the description below separately describes the operator functions and the administrator functions, although it can be appreciated that there may be some overlap of functions and some of the administrator functions may be implemented by the operator, and vice-versa.

### Operator Functions

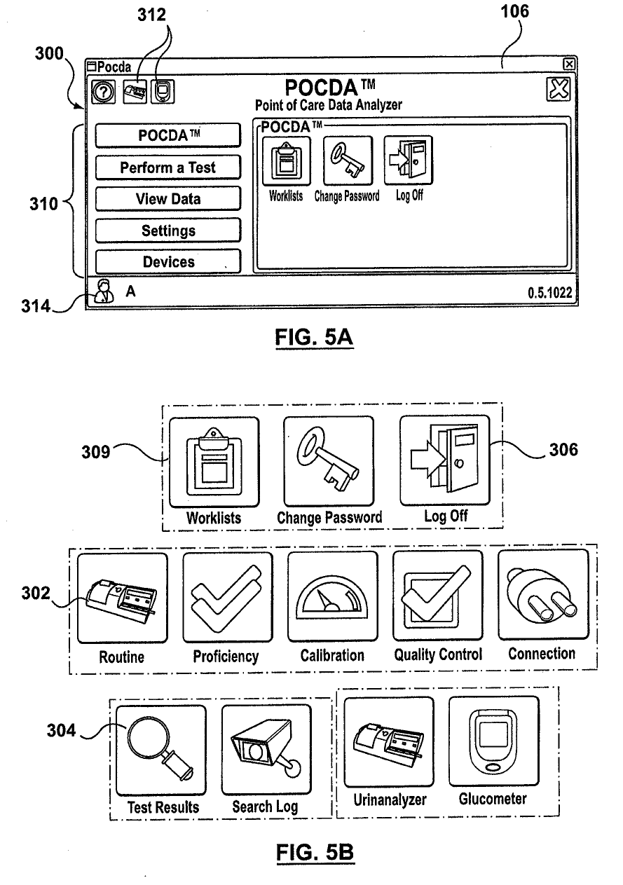

Reference is now made to FIG. 5A, which shows a diagrammatic view of a main menu graphical user interface 300 for the data managing device 100 rendered on the display screen 106 by controller 102, after successful login by an operator. A user indicator 314 displays the name of the present user. It can be appreciated that other indicators may be used to indicate the present user and are not limited to these letters, such as the user's name or employee number.

As shown in FIG. 5A, the user interface 300 may display a menu 310. Selection of one of the options from the menu 310 may bring up a series of further user selectable icons related to that option. Some icons may be hidden from the operator depending on the restrictions configured in the device 100. Also shown in FIG. 5A are two device indicators 312 representing a urine analyzer 12, and a glucose monitoring device 14. One such device indicator 312 may be highlighted, representing the current device 20 being used by the operator. By selecting these indicators 312, the operator may manually change the active device.

Reference is now made to FIG. 5B, which shows some of the user selectable icons which may be displayed after selection from the menu 310 (FIG. 5A). As shown, the user selectable icons include a testing button 302, a results button 304, and a logout button 306. Selection of the results button 304 permits the user to view results based on different criteria such as by date, operator, patient, etc. Turning briefly to FIG. 15, selection of the results button 304 may for example display the results user interface 400, as shown. Selection of the logout button 306 causes the device 100 to logout the present user. Another user may thereafter login in the same manner as explained above, using the login user interface (not shown).

When selecting the testing button 302, the user interface that is displayed on the display screen 106 depends on the settings as configured by the administrator. Generally, the administrator may set specific control tests to be performed in the diagnostic instrument 20 (FIG. 1) before testing is performed. The control tests may include calibration tests, quality control tests, proficiency tests, and the like. These control tests may be performed based on criteria or a trigger event set by the administrator, for example based on time frequency, change of user/operator, change of strip lot, change of strip bottle, or change of diagnostic device. In some example embodiments, if the trigger event requires the control test to be performed, the usual testing of the sample specimen is disabled or locked until such control test is performed, and the user interface displayed on the display screen 106 is of the specific control test instead. The configuration of these features by the administrator is explained in greater detail below.

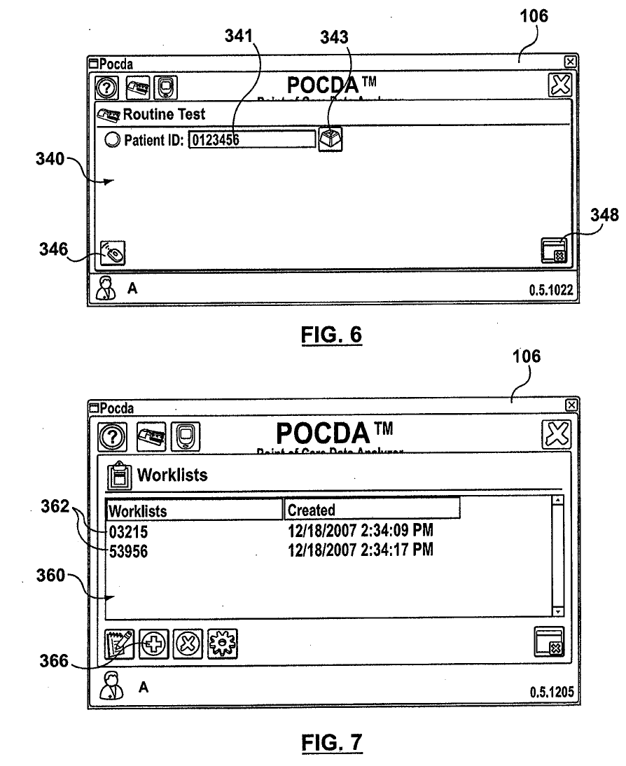

Reference is now made to FIG. 6, which shows a testing graphical user interface 340, which may be displayed on the device 100 upon selection of the testing button 302 (FIG. 5B) and when there is no trigger event occurring. In other words, the testing graphical user interface 340 may be used to perform regular/usual testing of the sample specimen. As shown, the testing graphical user interface 340 includes user selectable buttons such as an upload button 346 and a back button 348. Generally, selection of the back button 348 returns the present user interface to the previously displayed user interface, as appropriate.

Also shown is a patient ID field 341 which may be used to input the patient identification, wherein the patient identification may comprise of letters, numbers, symbols, or a combination thereof. The patient ID field 341 may for example be populated by using a keyboard 118 (FIG. 3), a touchscreen keyboard (not pictured) which appears when the operator presses the keyboard button 343, the barcode reader 116 (FIG. 3), or for example by downloading from the server device 18 through the network 16. Selection of the upload button 346 causes the device 100 to retrieve the patient profile corresponding to the patient ID.

Referring back to FIG. 5B, the worklists button 309 will now be explained in greater detail, referring now to FIG. 7, which shows a graphical user interface 360 displaying a worklist menu. As shown, a user may select from a number of worklists 362 from the user interface 360. In the example shown, there are two worklists 362, named 03215 and 53956 respectively. Worklists 362 may also be pre-loaded into storage 108 (FIG. 3). Generally, when a user is performing patient tests using the worklists 362, they may stop using the worklist 362, and may resume working on it at a later date. Also shown is the add button 366, which allows a user to create a new worklist 362 or download existing worklists to the device 100 via the network 16 (FIG. 1), for example from the server device 18 (FIG. 1).

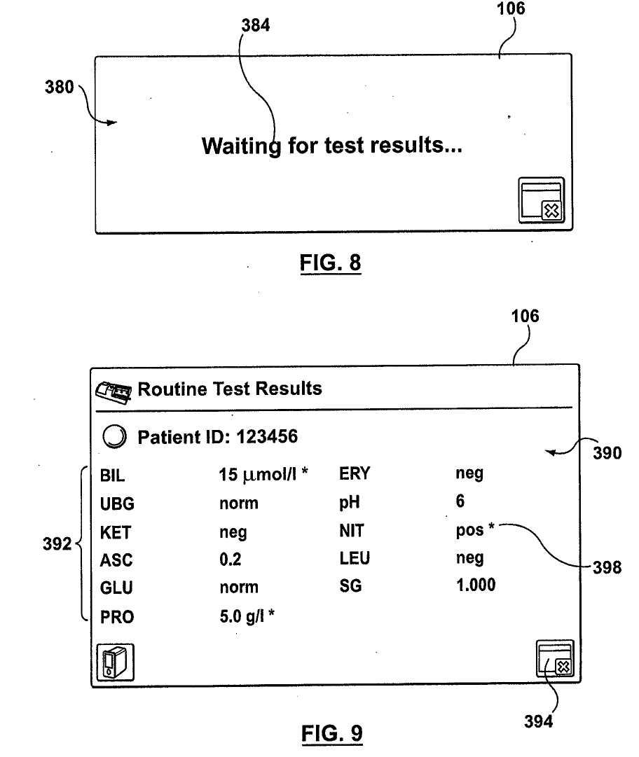

Reference is now made to FIG. 8, which shows a graphical user interface 380 which is displayed after a patient ID is inputted (as explained above) or when an appropriate worklist 362 (FIG. 7) is chosen. The device 100 may send appropriate protocols or prompts to the diagnostic instrument 20 to indicate that sample testing will be taking place. A user may perform the test as required by the diagnostic instrument 20. The user interface 380 thereafter displays a status message 384, indicating that the urine analyzer 12 is performing sample testing and data analysis on the sample specimen. In other embodiments, the device 100 may provide audible instructions, for example through a speaker (not shown), or written instructions on the user interface 380 on how to perform the test.

Once testing is complete, referring now to FIG. 9, the device 100 displays a user interface 390 displaying the results of the test. The results 392 are listed on the user interface 390, showing suitable parameters based on the specific sample test, in this instance a urine analysis test. Any values with a pathological significance (positive results) are indicated by an indicator such as an asterisk (*) 398, a change in colour (for example yellow), or any other appropriate indicator. The user may then press the OK button 394, which stores the results 392 in the appropriate worklist 362 (FIG. 7). In some example embodiments, the results 392 also are sent through the network 16 (FIG. 1) to the server device 18 (FIG. 1).

Referring again to FIG. 5B, referring again to the selection of the testing button 302, in some embodiments the device 100 may be preconfigured by an administrator to display a different user interface than the user interface 340 shown in FIG. 6, based on a control test being required. For example, the device 100 may be required to perform a calibration test, a quality control test, or a proficiency test.

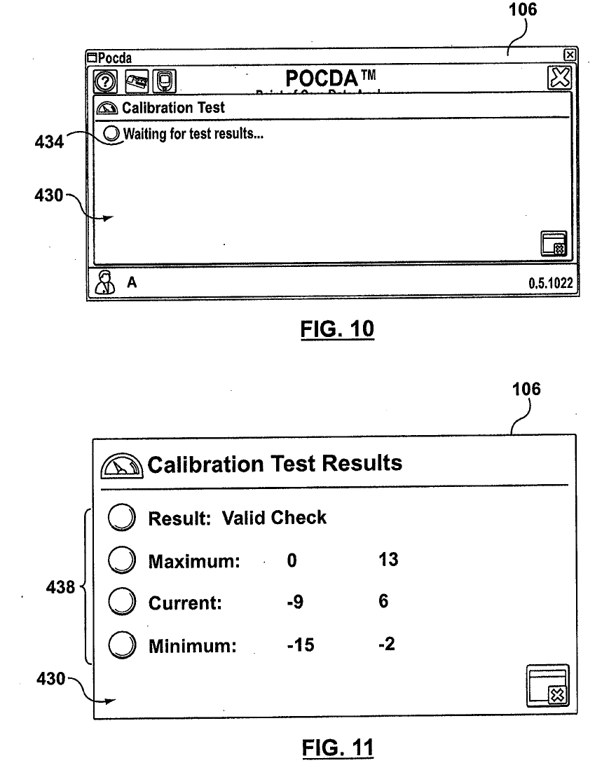

Referring now to FIG. 10, if the device 100 requires a calibration test, the calibration user interface 430 is displayed when the testing button 302 (FIG. 5B) is selected. As well, the regular/usual sample testing described above may become disabled or at least temporarily inaccessible. Generally, the calibration feature checks if the diagnostic instrument 20 (FIG. 1) is working properly, i.e., the results from a control strip are within acceptable parameters. The user interface 430 displays a status message 434, indicating that the device 100 is waiting for the calibration result from the diagnostic instrument 20 (FIG. 1). A user may then perform the calibration as required by the diagnostic instrument 20 (FIG. 1). If the diagnostic instrument 20 has no calibration feature, for example the glucose meter 14 (FIG. 1), the user interface 430 may not appear at all.

FIG. 11 shows the user interface 430 displaying results of the calibration test. As indicated, the user interface 430 will state that calibration was valid/successful (or not successful, as appropriate). Also shown are the calibration results 438, indicating the maximum, minimum, and current levels of a given or predetermined parameter.

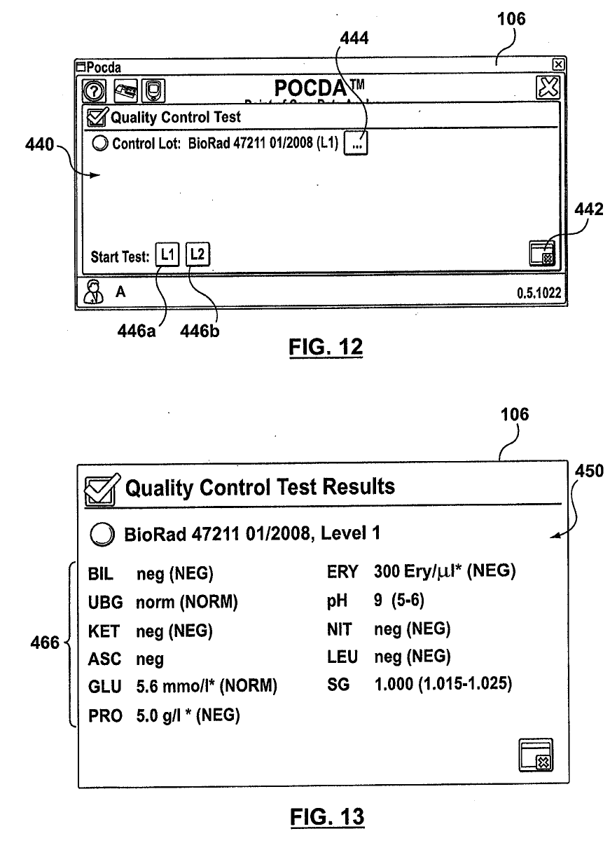

Referring now to FIG. 12, if the device 100 requires a quality control test, the quality control user interface 440 is displayed on the display screen 106 when the testing button 302 is selected. A quality control test generally ensures validity/quality of the test strips being used. A quality control test is usually performed by treating a test strip with a level 1 (low) reagent and a level 2 (high) reagent in order to determine that the test strips of the same lot or bottle generate expected parameter values. The expected parameters or reference values may for example be input by an administrator (as explained in greater detail below) or may be downloaded into the device 100 via the network 16 (FIG. 1). The user may select a control solution or edit the present control solution by selecting it from the pick lot button 444, or may exit the user interface 440 by pressing the cancel button 442. Selection of the pick lot button 444 may for example display another user interface (not shown) to permit the user to select or change the presently used quality control solution. The user performs the test by pressing the level 1 button 446a or the level 2 button 446b. The user treats the test strip with the level 1 solution and inputs into the input component of the urine analyzer 12 (FIG. 1). A status message 446 indicates that the device 100 is waiting for level 1 test results. After this, these steps are repeated for level 2 testing (not shown).

FIG. 13 shows the user interface 450 displaying results of the quality control test. As indicated, the user interface 450 will state that calibration has failed (or was successful, as appropriate). Also shown are the quality control results 466. Any quality control results 466 which do not correspond to the reference values are indicated by an indicator such as an asterisk (*) 464, a change in colour (such as yellow), or any other appropriate indicator.

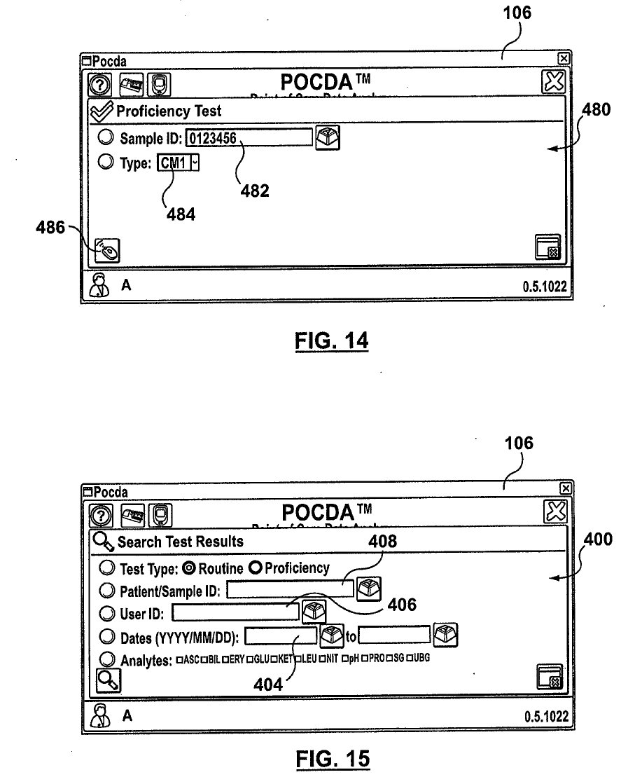

Referring now to FIG. 14, if the device 100 requires a proficiency test, the proficiency user interface 480 is displayed on the display screen 106 when the testing button 302 (FIG. 5B) is selected. Generally, different proficiency tests are performed based on a specific control solution for a laboratory or other facility to meet its regulatory requirements. The control solution is tested and the results are sent to an appropriate regulatory body for approval of the results. As shown on the user interface 480, the user may select any number of proficiency tests to perform, by selecting one from the proficiency test dropdown 484. The user may populate the sample ID field 482 in a manner similar to inputting of the patient ID field (FIG. 6), described above. After inputting the sample ID field 482, the test strip is treated with the specific sample and input into the diagnostic instrument (FIG. 1). By pressing the upload button 486, the results are sent to the server device 18 or to the regulatory body via the network 16. The results may also be stored in storage 108 (FIG. 3). The results may also be printed using the printer 142 (FIG. 3), and filed or mailed to the regulatory body, etc.

Referring again to FIG. 5B, accordingly, the above description generally describes some of the possible user interfaces that may be displayed when a user selects the testing button 302.

Referring still to FIG. 5B, the user may select the results button 304. Referring now to FIG. 15, the selection of the results button 304 causes the device 100 to display a results user interface 400, which may for example assist a user in searching and subsequently viewing of different test results. As shown, a user has a number of selectable options for viewing of results, including a view between dates field 404, view by operator field 406, and view by patient field 408. The insertion of data in these fields results in displaying of a results list (not shown) of the results stored in storage 108 (FIG. 3) based on the respective criteria. The selection of any item on the results list (not shown) will result in viewing of the results, for example on an interface similar to user interface 390 (FIG. 9).

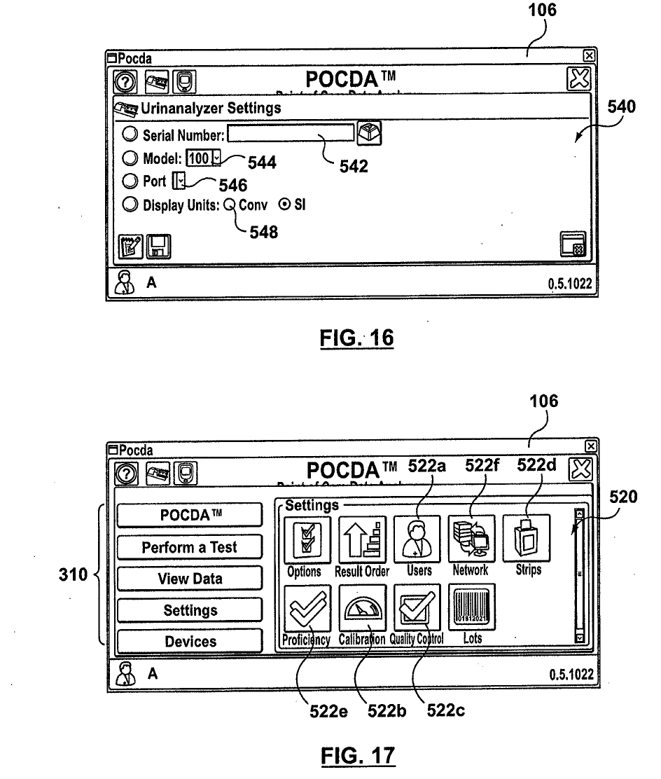

Referring again to FIG. 5B, the user may select the device settings button 308 for their diagnostic instrument 20. Referring now to FIG. 16, the device 100 displays a user interface 540 having a number of configurable parameters related to the specific diagnostic instrument. The interface shown in FIG. 16 is for a urine analyzer 12, but it may be adjusted for other devices. As shown, the parameters include urine analyzer serial number 542, model number 544, communications port 546, display units 548 (which may be conventional or SI),

### Administrator Functions

Reference is now made to FIG. 17, which displays the contents of the Settings menu when selected from the menu 310 (FIG. 5A). The device 100 in response displays a user interface 520 having a number of user selectable setup icons 522. As shown, the icons 522 include operators icon 522a, calibration icon 522b, quality control icon 522c, strips icon 522d, proficiency icon 522e, network icon 522f.

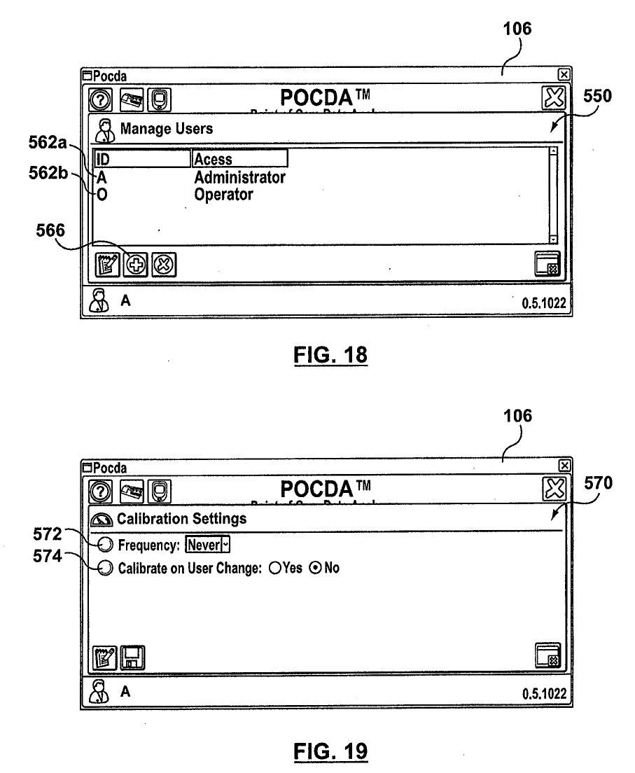

By selecting the operators icon 522a (FIG. 17), referring now to FIG. 18, the device 100 displays a user interface 550 having icons corresponding to each of the users 562. As shown, the users 562 are Administrator (A) 562a and the Operator (O) 562b. Selection of the icons corresponding to the user 562 allows the administrator to configure settings relating to the user, including change in user name and password, deleting a user, and upgrading accessions rights for the user. Selection of the add button 566 allows the administrator to add a new user and corresponding password.

By selecting the calibration icon 522b (FIG. 17), referring now to FIG. 19, the device 100 displays a calibration user interface 570 for configuring the criteria or trigger event for performing calibration of the urine analyzer 12 (FIG. 1). Some example trigger events are frequency 572, and user change 574. When any of these trigger events require calibration on the device 100, the device 100 may thereby be disabled from performing regular/usual sample testing of strips. Frequency 572 of calibration may for example be none, daily, weekly, monthly or any suitable interval. User change 574 may be yes/no toggled to require calibration each time a user logs into the device 100 (or any other indicator of user change).

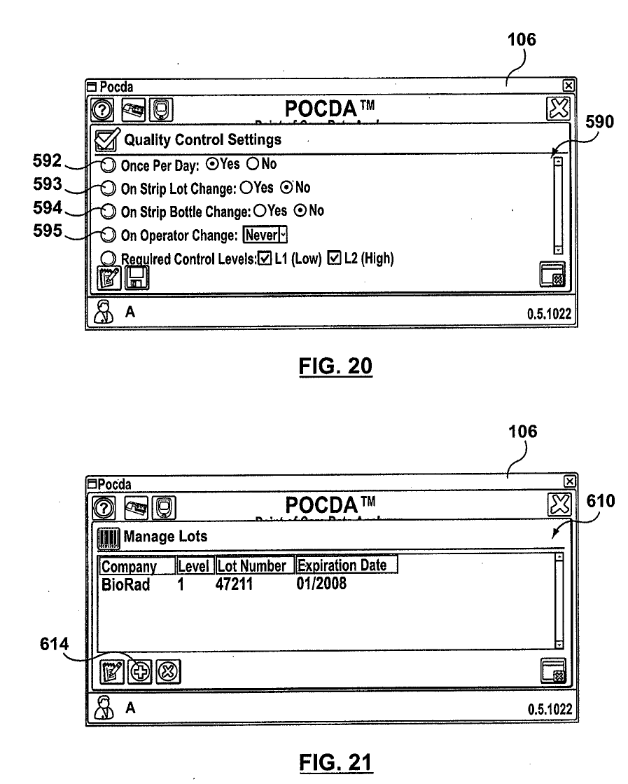

By selecting the quality control icon 522c (FIG. 17), referring now to FIG. 20, the device 100 displays a quality control interface 590 for configuring the criteria or trigger event for performing quality control of the urine analyzer 12 (FIG. 1). Some example trigger events are once per day 592, strip lot change 593, bottle change 594 and operator change 595. When any of these trigger events require calibration on the device 100, the device 100 may thereby be disabled from performing regular/usual sample testing of strips. Once per day 592 may be yes/no toggled to require calibration every day. Strip lot change 593 may be yes/no toggled to require calibration each time a new lot of strips is used in the device 100. Bottle change 594 may be yes/no toggled to require calibration each time there is a change of strip bottle. Operator change 595 may be set to Never, Daily, or Always. If set to Always, the device 100 will require calibration each time a user logs into the device 100 (or any other indicator of user change). If set to Daily, the device 100 will require calibration the first time each user logs into the device 100 each day.

Selection of the lots button 598 allows the administrator to perform selection and editing of the quality control lots. Referring now to FIG. 21, a user interface 610 shows the available lots. As shown, the Biorad lot 47211 may be selected by the user. The user may also select the add button 614 to add a new lot, by manually inputting the values or by downloading the lot values from the network 16 (FIG. 1).

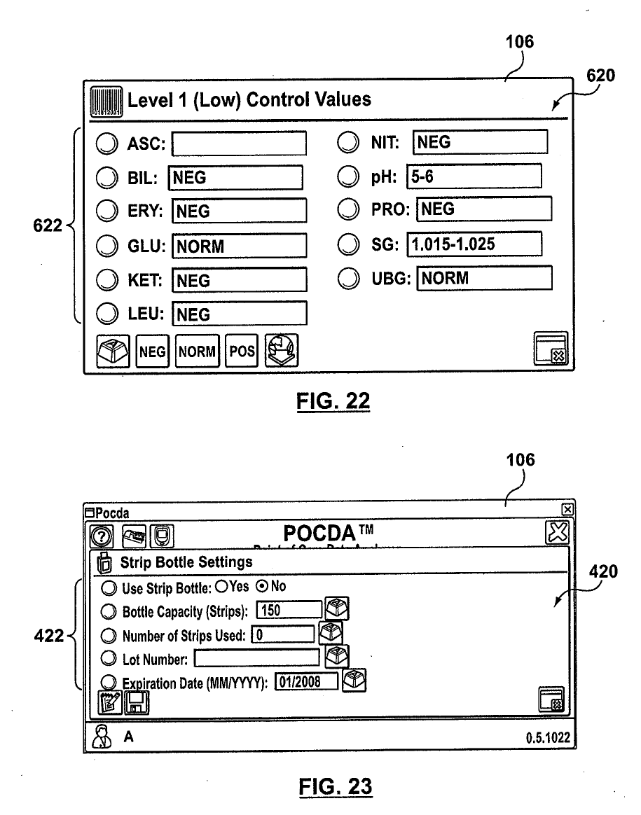

Referring now to FIG. 22, an administrator or user may edit the particular lot control parameters 622 for the given quality control solution using the user interface 620. The lot control parameters 622 may then be saved in storage 108 (FIG. 3).

Referring again to FIG. 17, selection of the strips icon 522d results in the device 100 displaying the strips user interface 420 (FIG. 23), which may display a list of strips parameters 422. As indicated, strip parameters including Use Strip Counter, Number of Strips in a Bottle, Number of Strips Used, Lot Number, and Expiry Date. By selecting the edit button 426, the user may edit the list of strips parameters 422, as appropriate.

Referring still to FIG. 17, selection of the proficiency icon 522e results in a user interface (not shown) for editing the triggering event for performing proficiency tests on the urine analyzer 12 for regulatory purposes. Examples of trigger events include frequency, change of user/operator, change of strip lot, change of strip bottle, or change of diagnostic device. When any of these trigger events require proficiency testing on the device 100, the device 100 may thereby be disabled from performing regular/usual sample testing of strips.

Referring still to FIG. 17, selection of the network icon 522f allows the administrator to perform network related functions, including for example modify connectivity settings, configure the server device 18 (FIG. 1), maintain passwords, and configure file transfer settings.

### Server Device

Referring again to FIG. 1, the server device 18 may for example be a conventional personal computer connected to the network 16 and in communication with the data managing device 100. The server device 18 may have user interface screens corresponding to those of the data managing device 100, and may have additional user interface screens with additional functionality. The server device 18 may also have additional storage capabilities for receiving of data from the data managing device 100, for storage and subsequent manipulation of data generated from the diagnostic instruments 20. The server device 18 may also periodically synchronize data such as patient files contained in storage 108 (FIG. 3).

The server device 18 may also be configured to send data received from the data managing device 100 through the network 16 to a remote device (not shown), for example to a third party such as a regulatory agency (not shown).

Although the device 100 has been described mainly in the context of urine analyzers, embodiments of the device 100 could also be suitably configured to be applied to different types of diagnostic instruments.

While the invention has been described in detail in the foregoing specification, it will be understood by those skilled in the art that variations may be made without departing from the scope of the invention.

What is claimed is:

1. A data managing device for connection to a diagnostic instrument, the diagnostic instrument having an input component for receiving of sample specimen, an analyzing component for generating data based on testing of sample specimen, a communications component, the diagnostic instrument being responsive to specific protocols communicated through the communications component for controlling of the diagnostic instrument, the data managing device comprising:
a controller for controlling the operation of the data managing device;
a casing for housing of the controller;
a display in communication with the controller and sealed with respect to the casing;
a user input coupled to the controller and sealed with respect to the casing;
a communications interface in communication with the controller for communication with the communications component of the diagnostic instrument;
the controller configured to control the diagnostic instrument by communicating the specific protocols to the diagnostic instrument over the communications interface.

2. The data managing device of claim 1, further comprising electronic storage accessible by the controller, the storage having stored preconfigured information including the specific protocols of the diagnostic instrument, wherein the controller, based upon a connection of the communications interface to the communications component of the diagnostic instrument, is configured to access the specific protocols for controlling of the diagnostic instrument over the communications interface.

3. The data managing device of claim 1, wherein the specific protocols include instructions for testing of sample specimen in the diagnostic instrument, the controller configured to instruct the diagnostic instrument to generate data based on testing of the sample specimen, and the controller configured to receive data based on testing of the sample specimen over the communications interface.

4. The data managing device of claim 1, wherein the diagnostic instrument is configured for control testing of the diagnostic instrument, the specific protocols include instructions for performing the control testing of the diagnostic instrument, and the controller configured to instruct the diagnostic instrument to perform the control testing of the diagnostic instrument.

5. The data managing device of claim 4, wherein the controller determines that the control testing of the diagnostic instrument is required based on predetermined criteria.

6. The data managing device of claim 5, where upon in determining that the control testing of the diagnostic instrument is required, the controller disables testing of sample specimen in the diagnostic instrument until control testing of the diagnostic instrument is completed.

7. The data managing device of claim 1, wherein the controller is configured to communicate data based on testing of sample specimen through the communications interface to a server device over a network.

8. The data managing device of claim 1, wherein the user input includes a touchscreen on the display.

9. The data managing device of claim 1, wherein the casing, the display, and the user input are hermetically sealed.

10. The data managing device of claim 1, wherein the diagnostic instrument includes a urine analyzer.

11. The data managing device of claim 1, wherein the diagnostic instrument is a handheld electronic device.

12. A method of facilitating data management in a diagnostic instrument, the diagnostic instrument having an input component for receiving of sample specimen, an analyzing component for generating data based on testing of sample specimen, a communications component, the diagnostic instrument being responsive to specific protocols communicated through the communications component for controlling of the diagnostic instrument, the method comprising:
providing a data managing device for connection to the diagnostic instrument;
storing at the data managing device the specific protocols of the diagnostic instrument;
coupling the data managing device to the diagnostic instrument; and
accessing, based upon the coupling of the data managing device to the diagnostic instrument, the specific protocols for controlling of the diagnostic instrument over the communications interface.

13. The method of claim 12, wherein the method further comprises treating a test strip with a sample specimen and applying the test strip to the input component of the diagnostic instrument.

14. A method of facilitating data management in a diagnostic instrument, the diagnostic instrument having an input component for receiving of sample specimen, an analyzing component for generating data based on testing of sample specimen, a communications component, the diagnostic instrument being responsive to specific protocols communicated through the communications component for controlling of the diagnostic instrument, the method comprising:
providing a data managing device for connection to the diagnostic instrument, the data managing device configured for providing instructions to the diagnostic instrument for testing of sample specimen;
determining in the data managing device that control testing in the diagnostic instrument is required based on predetermined criteria; and
disabling testing of sample specimen in the diagnostic instrument.

15. The method of claim 14, wherein the method includes, after disabling testing of sample specimen in the diagnostic instrument:
performing control testing in the diagnostic instrument; and
enabling testing of sample specimen in the diagnostic instrument.
